(19) United States
(12) Patent Application Publication
(10) Pub. No.: US 2009/0318392 A1
(43) Pub. Date: Dec. 24, 2009

(54) BIOFLUID METABOLITE PROFILING AS A TOOL FOR EARLY PREDICTION OF AUTOIMMUNITY AND TYPE 1 DIABETES RISK

(75) Inventors: Matej Oresic, Espoo (FI); Olli Simell, Raisio (FI); Tuulikki Seppänen-Laakso, Espoo (FI); Marko Sysi-Aho, Helsinki (FI)

Correspondence Address: YOUNG & THOMPSON, 209 Madison Street, Suite 500, Alexandria, VA, 22314, US

(73) Assignee: ZORA BIOSCIENCES OY, Espoo (FI)

(21) Appl. No.: 12/441,197

(22) PCT Filed: Sep. 10, 2007
(86) PCT No.: PCT/FI2007/000227

§ 371 Date: Jun. 5, 2009

Related U.S. Application Data
(60) Provisional application No. 60/844,357, filed on Sep. 14, 2006.

(51) Int. Cl. A61K 31/66; G01N 33/92; A61K 31/14

(52) U.S. Cl. 514/114; 436/71; 436/131; 514/642

(57) ABSTRACT

The invention concerns a method for diagnosing a child's susceptibility for developing type 1 diabetes by using a serum metabolite as biomarker. The invention concerns also a method for prevention of the onset of type 1 diabetes in a child.

### FIELD OF THE INVENTION

This invention relates to a method for early diagnosing of a child's susceptibility for developing type 1 diabetes. Furthermore, the invention also relates to a method for the prevention of type 1 diabetes in a child diagnosed as susceptible for developing type 1 diabetes.

### BACKGROUND OF THE INVENTION

The publications and other materials used herein to illuminate the background of the invention, and in particular, cases to provide additional details respecting the practice, are incorporated by reference.

Type 1 diabetes is an autoimmune disease, in which the body's own immune system attacks the β cells in the islets of Langerhans of the panceras, destroying them or damaging them sufficiently to reduce or eliminate insulin production.

During the past 10-50 years the incidence of type 1 diabetes, the most common metabolic-endocrine disease of children, has increased for unknown reasons in almost all western countries. Development of β cell-specific autoantibodies, believed to occur together with selective functional impairment and ultimate destruction of the insulin-producing β cells in the pancreatic islets of Langerhans, commonly precedes the onset of overt type 1 diabetes by months to years. The unknown initiators of autoimmunity and the poorly understood mechanisms supporting progression towards β cell failure not only hinder estimation of the absolute disease risk and time of onset of the disease in genetically susceptible individuals, but also hamper discovery of effective prevention.

### OBJECTS AND SUMMARY OF THE INVENTION

An object of the present invention is to provide a method for early diagnosis of a child's susceptibility for developing type 1 diabetes.

Particularly, the object is to provide a method for diagnosing the child's risk of developing type 1 diabetes in months or years before the clinical onset of the disease, preferably even before the emergency of autoantibodies in the child's serum. A particular object is to provide a method for diagnosing even a newborn child's risk of developing type 1 diabetes at a later stage.

Furthermore, one object of this invention is to provide means for prevention of the onset of type 1 diabetes in a child diagnosed as susceptible for developing type 1 diabetes.

To overcome the obstacles relating to established methods, we launched Type 1 Diabetes Prediction and Prevention study (DIPP), a large birth cohort study, in Finland in 1994¹. The type 1 diabetes risk- and protection-associated HLA-alleles were first analyzed in cord blood of newborns after parental informed consent. Children carrying increased genetic risk were then frequently examined to discover when diabetes-associated autoantibodies emerged, or clinical diabetes developed. During the 11.5 years of the study, over 100,000 newborns have been screened, over 450 have developed multiple autoantibodies indicating markedly elevated disease risk, and 138 of those remaining in the study have so far progressed to clinical diabetes, providing a unique series of samples for studies of disease pathogenesis and prediction.

Serum patterns of metabolites at least to some extent reflect homeostasis of the system, and changes in specific metabolite groups may system responses to environmental or genetic alterations or interventions². Metabolomics platform, applicable to all species, follows a time-response, and has capability for high sample throughput. The metabolic phenotype is also affected by environmental factors such as nutrition and gut microbiota^{3,4}, which are of particular relevance to complex diseases such as type 1 diabetes, believed to be affected both by genetic factors and the environment⁵. With today's analytical and information technologies for handling of large volumes of data, the metabolomics approach has become increasingly feasible. Therefore, metabolomics may provide powerful tools for characterization of e.g. complex phenotypes and biomarkers for selected physiological and pathological responses^{6,7}.

Thus, in its broadest aspect, this invention relates to a method for diagnosing a child's susceptibility for developing type I diabetes, wherein said method comprises the steps of

i) determining the concentration of at least one serum metabolite in the child to be diagnosed,

ii) comparing the serum concentration of said metabolite to the serum concentration of the same metabolite in a control group of healthy children, and

iii) using a concentration difference between the child to be diagnosed and the control group as a biomarker indicative of the child's susceptibility for developing type I diabetes.

In another aspect, this invention relates to a method for prevention of the onset of type 1 diabetes in a child, said child having been diagnosed according to this invention, as susceptible for developing type I diabetes, said method comprising subjecting said child one or more measures preventing the onset of diabetes.

### BRIEF DESCRIPTION OF THE DRAWINGS

FIG. 1. Design of the DIPP study and the sample selection for metabolomics. (a) DIPP study design. (b) Autoantibody profile of a child who developed diabetes at age of 9 years. (c) Samples included in the study. (d) Design of analysis and the main questions addressed in this paper.

FIG. 2. Two-dimensional Sammon's mapping of all samples in the Oulu batch. Total 518 samples included, with 186 identified lipids as variables. Four different potential confounding factors are visualized following the mapping. (a) Individual ID, (b) gender, (c) age, and (d) sample age.

FIG. 3. Profiles of selected ether linked phosphocholine species from DIPP Turku batch. (a) Longitudinal profiles of GPCho(36:2e). (b) Longitudinal profiles of GPCho(40:4e). (c) GPCho(36:2e) levels at age of 1 year. Only one sample per individual included, nearest to 1 year of age. (d) GPCho(36:2e) levels at age of 3 years. (e) GPCho(36:2e) levels at age of 6 years.

FIG. 4. Cord blood lipid profiles. (a) The score plot reveals differences in lipid profiles between the progressors and majority of non-progressors at birth. (b) The loadings reveal the differences are attributed to phospholipids. (c) The ether linked phosphocholine GPCho(36:2e) is not different between progressors and non-progressors. (d) Total phosphocholine level, calculated as a sum of concentration of all ester linked glycerophosphocholine molecular species.

FIG. 5. Lipid profiles within the 6 month interval before and after seroconversion. (a) Changes in lipid profiles are detected using PLS/DA analysis. (b) Lysophosphatidylcholine species LysoGPCho(18:0), known to be associated with inflammation, is upregulated in progressors prior to seroconverion, i.e. emergence of first autoantibodies. (c) Ether linked phosphoethanolamine species GPEth(38:1e) is upregulated after seroconversion in progressors.

FIG. 6. Summary of findings in context of Type 1 Diabetes pathogenesis.

FIG. 7. Pathway showing the synthesis choline plasmalogens from DHAP.

FIG. 8. Changes in ether phosphatidylcholine levels in progressors between the ages of 1.5 and 5 years Only one sample per individual, drawn closest to the age of 1.5 or 5 years is included. (Panel A). The exact fatty acid position (i.e., sn1 vs. sn2) and double bond locations were not confirmed. Panel B shows box plot of GPCho(O-18:1/16:0) concentrations for progressors and non-progressors. The box contains the middle 50% of the data. The upper edge (hinge) of the box indicates the 75th percentile of the data, and the lower hinge indicates the 25th percentile. Illustrative longitudinal profiles for two progressors and two non-progressors (Panel C). Panel D lists lysophosphatidylcholine level changes between progressors and non-progressors within a 9-month period preceding seroconversion and soon thereafter. The non-progressor selected time points were closest to those of matched progressors.

FIG. 9. Early age differences between progressors and non-progressors for the ether phosphatidylcholine GPCho(O-18/18:2). Levels for children with ages between 315 and 405 days (1 year) are shown in Panel A, with ages between 630 and 810 days (2 years) in Panel B, and with ages between 1980 and 2340 days (6 years) in Panel C. Only one sample per individual included, obtained nearest to the indicated age. Panel D shows the longitudinal profiles of GPCho(O-18:0/18:2) for subjects from batch 1.

FIG. 10. Early age differences between progressors and non-progressors for the ethanolamine plasmalogen GPEtn(O-18:1(1Z)/20:4). The plasmalogen levels are shown for children with ages between 315 and 405 days (Panel A) and with ages between 1980 and 2340 days (Panel B).

### DETAILED DESCRIPTION OF THE INVENTION

We hypothesized that abnormalities in serum metabolite profiles might precede onset of autoimmunity revealing pathways essential for type 1 diabetes development. We tested this hypothesis by screening during 11.5 years genetic diabetes risk in over 100,000 consecutive newborns and enrolling the children with genetic diabetes risk to tight follow-up. From over 8500 children continuing in the study, over 450 have produced multiple types of autoantibodies, and 138 have progressed to overt type 1 diabetes. The metabolite profiles in 1039 serum samples collected at 3- to 12-month intervals between birth and diabetes onset from a cohort of 47 children were compared with the profiles of 60 children who remained healthy and autoantibody negative. We observed marked differences in metabolite patterns in cord blood and later samples between progressors and permanently autoantibody-negative children. Metabolites protecting against oxidative stress and those related to inflammation differed strongly between the cases and the controls markedly earlier than the children seroconverted to autoantibody positivity. The findings imply that metabolomics can effectively be applied to screening of diabetes risk in infancy and early childhood, and suggest that poor protection against oxidative damage and inflammation plays important role in the pathogenesis of diabetes.

### Preferred Embodiments

The serum metabolite to be used as biomarker is preferably a metabolite protecting against oxidative stress and/or inflammation. In this case, a decreased concentration thereof in the child to be diagnosed, compared to the control group of healthy children, is indicative of the child's susceptibility for developing type I diabetes.

The wording “decreased concentration” shall be understood to mean that the level of the biomarker in the child belonging to the risk group may be up to 80% of the level of the same biomarker in healthy controls. However, typically the level in the risk group is at highest 75%, more typically 65 . . . 50% of the level in the controls.

In one preferred embodiment, the biomarker is total phospholipids, one or more ester linked phosphocholines, or total ester linked phosphocholines. In all these cases, the biomarker is preferably determined already in newborn children, for example by cord blood analyses. In a particularly preferred embodiment, the child is a newborn child and child's level of total ester linked phosphocholines being about 80% or less of the mean level for the control group is used as indicative of the child's susceptibility for developing type I diabetes.

In another preferred embodiment, the biomarker is one or more ether linked phosphocholines, such as (but not restricted to) GPCho (36:2e), GPCho (38:1e), GPCho (38:5e), GPCho (40:4e), CPCho (O-18:1/16:0), CPCho (O-18:1/16:1), CPCho (O-16:0/20:4), CPCho (O-18:1/20:4) or CPCho (O-18:0/18:2). Ether linked phosphocholines can be determined at a child age ranging from newborn to six years' age, preferably in the age of 1-2 years.

In a further preferred embodiment, the biomarker is an ethanolamine plasmalogen such as GPEtn (O-18:1(1Z)/20:4). This biomarker can be determined at a child age ranging from newborn to six years' age.

In a still further embodiment, the biomarker is an acid or a derivative thereof, a ketone, or an alcohol. As non-limiting examples of biomarkers in this group can be mentioned tryptophan, ribitol, pentanedioic acid, glycine, eicosanoic acid, 1,2,3-propanetricarboxylic acid, myristoleic acid, mannitol, creatinine, butanedioic acid, heptanoic acid, and 2-ketoglutaric acid methoxime. Out of these compounds, tryptophan, ribitol, pentanedioic acid, 1,2,3-propanetricarboxylic acid, creatinine enol and butanedioic acid are believed to be the most preferable ones.

The determination of the serum metabolite can be followed up at several ages of the child and the result is compared to control groups of the same age as the child to be diagnosed.

Also, several serum metabolites can be determined for the child to be diagnosed, and the levels are compared to the levels of said metabolites for control groups. All or some of such metabolites can be monitored over time.

The aforementioned monitoring of one or more serum metabolites can be combined with determination of genetic risk for development of type 1 diabetes and/or monitoring of emergence of autoimmunity in the child.

Preferably, the genetic risk for development of type 1 diabetes and/or the emergence of autoimmunity are followed by metabolite markers as progressive disease susceptibility detection.

Most preferably, the emergence of autoantibody markers in combination with the decreased ether linked phosphocholine levels are determined to identify individuals at higher risk of developing type 1 diabetes.

Once diagnosed at an early stage as belonging to the risk group, the onset of type 1 diabetes in the child can be prevented in many various ways. The preventing measure can be, for example, a nutritional intervention, an antioxidant therapy, or a stimulation of the biochemical synthesis of choline plasmalogens in the child, or any combination thereof.

As potential preventive measures for type 1 diabetes following from our results, are the use of nutritional interventions that are known to be safe, for example:
Choline supplements for mothers, particularly if any of parents carrying the risk genotype.
Choline supplements for children after birth if phosphocholine levels found low.
Choline plasmalogen supplements for children if phosphocholine levels found low at birth or low ether linked phosphocholine found low later on.

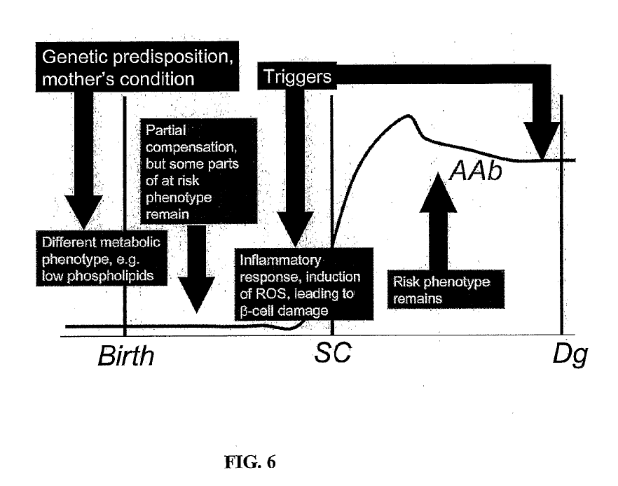

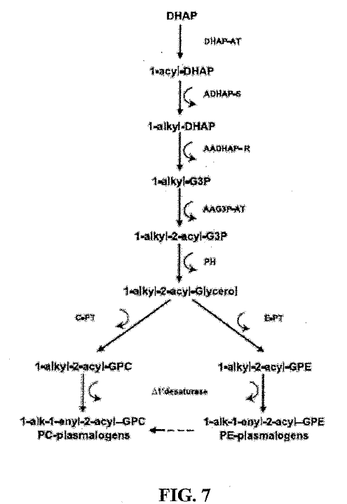

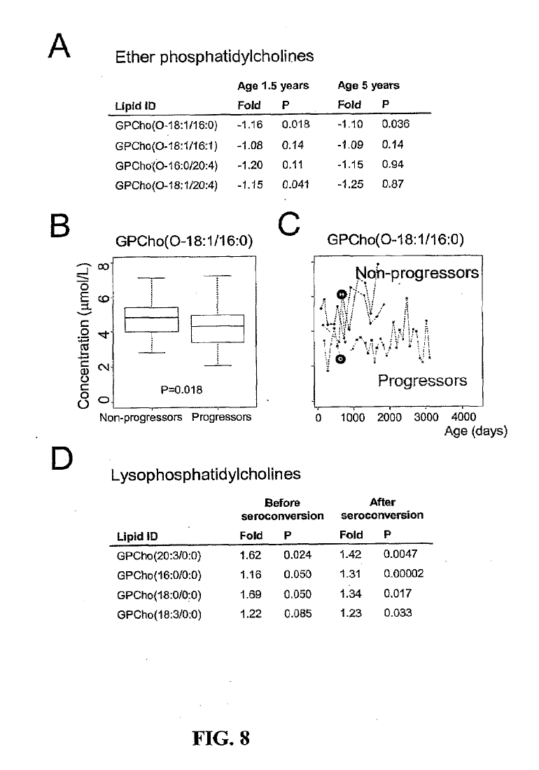

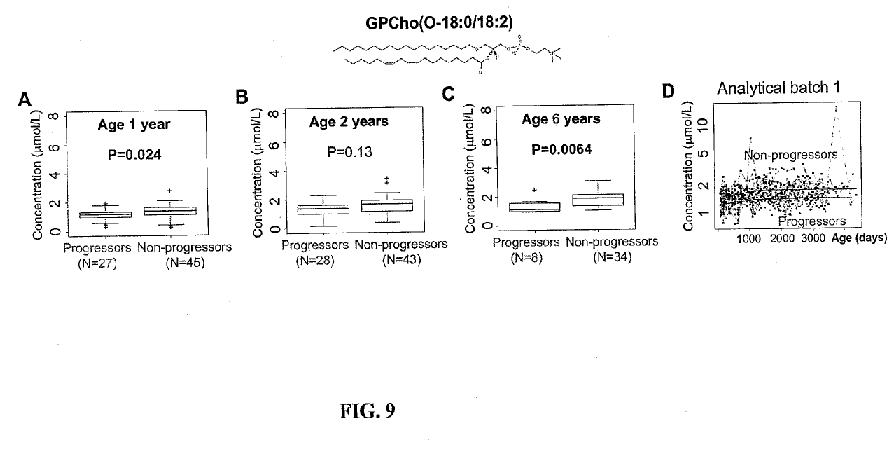

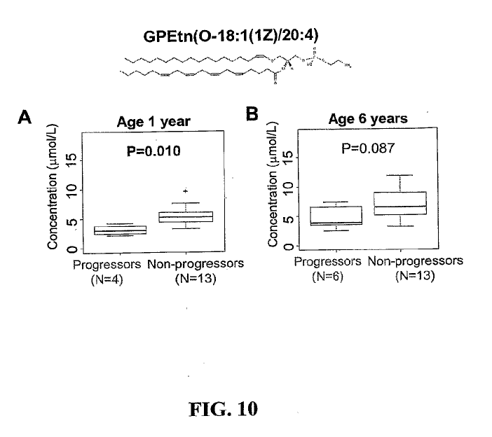

As a potential drug therapy, the antioxidant therapy is an option. As an alternative, stimulating the synthesis of endogenous antioxidants choline plasmalogens, found down-regulated in this invention, is one possible option. The pathway is shown in FIG. 7.

The invention will be illuminated by the following non-restrictive Experimental Section.

Experimental Section

We applied high-throughput metabolomics technologies to analyze the serum sample series collected from our study children between birth and development of overt diabetes (the progressors), and compared the findings to those obtained by studying sample series from control children matched for age, gender, genetic risk group and site of birth, but who never showed signs of diabetes-related autoimmunity or diabetes (the non-progressors). Patterns of serum lipids, water soluble compounds and serum albumin-bound metabolites differed in cord blood and samples collected during infancy and early childhood, totally changing the autoantibody-based prediction of type 1 diabetes. The identified metabolites and extrapolated pathways imply that factors protecting from oxidative stress and inflammation are highly important inhibitors of disease progression, providing potential targets for diabetes prevention.

Selection of Subjects

The DIPP project has been carried out in three cities in Finland with a combined annual birth rate of 11,000, representing almost 20% of births in Finland. The project was launched in Turku in November 1994; Oulu joined the study one year and Tampere three years later. HLA-DQB1 alleles *02, *0301, *0302, *0602 and *0603 were analyzed, and males positive for DQB1*02 were further typed for DQA1 alleles *0201 and *05 in the Turku cohort. The defined PCR amplified gene sequences were hybridized in solution with allele-specific, lanthanide chelate labelled oligonucleotide probes, and the hybridization products were detected using time-resolved fluorimetry (Victor, Wallac, Turku). By Jun. 6, 2006, 107,484 consecutive newborns and their older siblings had been screened, and around 8,000 children with genetic risk continued in the follow-up.

Our attempts to include to the screening analysis of polymorphisms of the insulin promoter region, CTLA4 and PTPN22 only marginally improved screening efficacy with rather poor cost-benefit ratio, forcing us to drop these assays from the routine screening, although we have continued using these assays for selected research purposes.

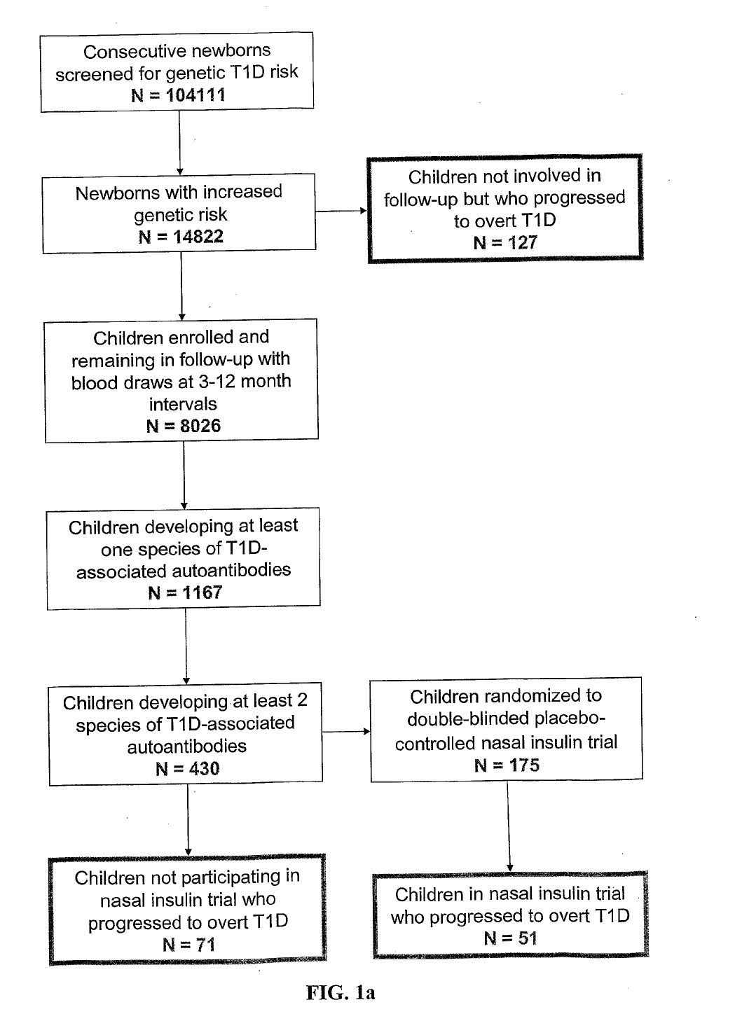

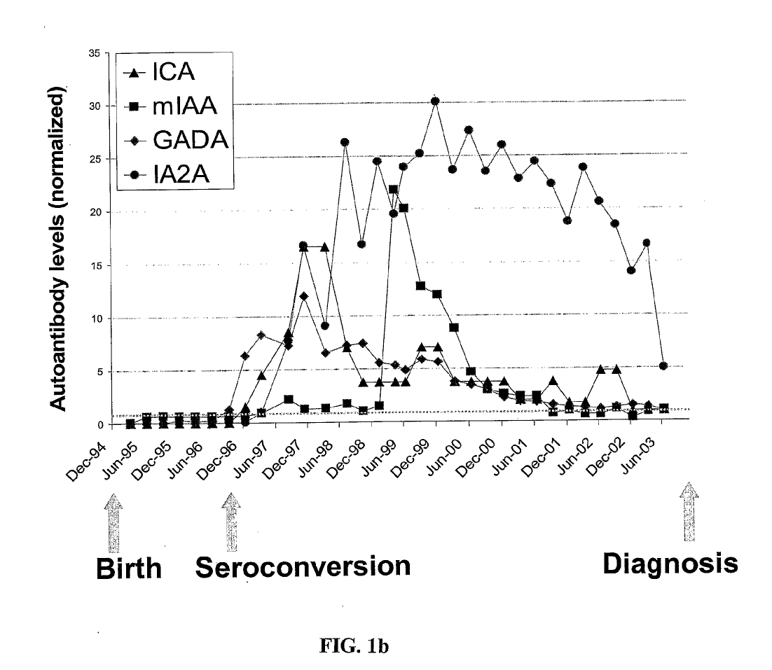

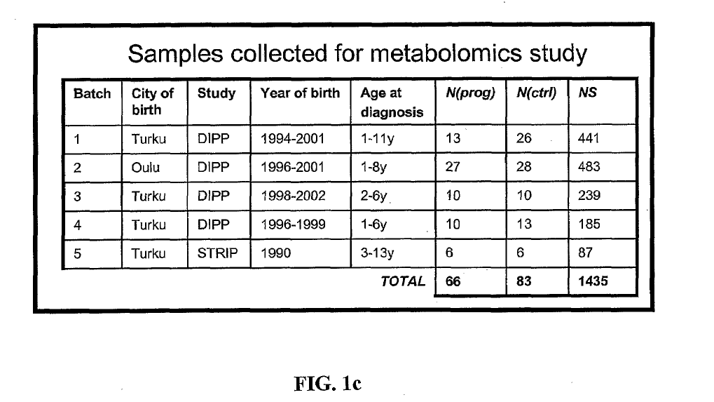

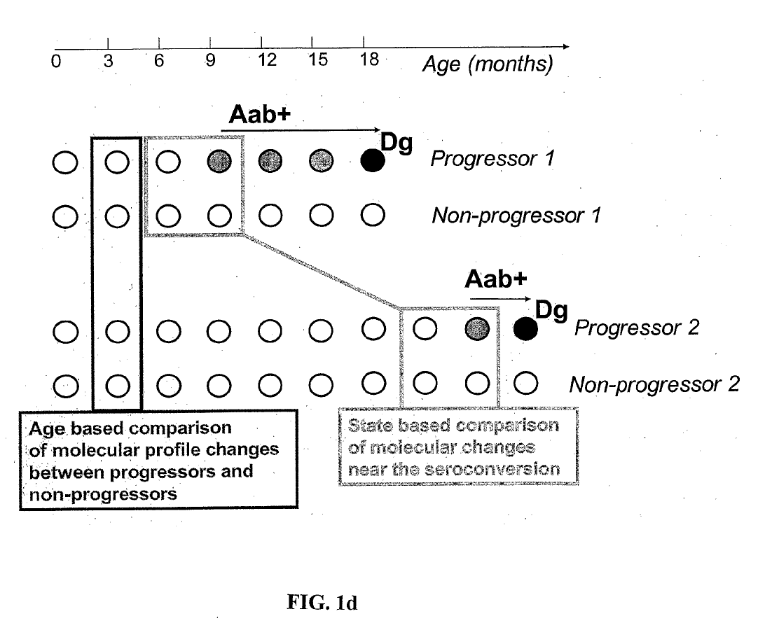

Of the study participants, 1445 had at least once been positive for autoantibodies against islet cells, insulin, glutamic acid decarboxylase or IA-2 protein. 516 of them had more than one type of antibodies, strongly increasing their likelihood of progressing to diabetes. Finally, 137 children developed overt type 1 during the follow-up (FIG. 1a-b). The majority of these 137 children developed as the first antibody IAA either alone or with ICA or GADA, while IA-2A was usually a late-appearing antibody. Some children developed diabetes rapidly within the first year of age, while other children with closely similar autoantibody patterns survived for years without progressing to overt diabetes (see e.g. FIG. 1b). Autoantibody values commonly varied markedly during the follow-up, but the values often slowly declined before development of clinical diabetes (see e.g. FIG. 1b).

Longitudinal serum collection from birth until disease onset (and possibly after) at 3-6 month intervals affords detailed study of disease pathogenesis and potential early mechanisms. The emergence of autoantibodies versus time is shown in FIG. 1b.

Subjects who progressed to overt Type 1 Diabetes were selected from the DIPP trial, matched by HLA genotype, gender, city and period of birth. Total 41 progressors and 54 non-progressors were selected, accounting to 950 samples (FIG. 1c). For the experiments and data analyses, the samples were further divided into two separate batches based on city of birth: Turku (13 progressors, 26 non-progressors) and Oulu (28 progressors, 28 non-progressors).

To compare metabolomics findings in the genetically defined DIPP population to a genetically non-defined group of children having prospectively collected sample series available between the ages of 7 months and post-puberty, we selected all six children who in the Special Turku Coronary Risk Factor Intervention Project for Children (STRIP) 8 had developed type 1 diabetes, and their six age- and sex-matched healthy controls from the same study (total 89 samples). The STRIP study comprised at recruitment 1062 children, out of whom over 700 continued in the study at child's age of 10.5 years. Only some of the children who developed diabetes carried HLA risk alleles, but all had multiple autoantibodies before clinical diabetes developed.

In our study of metabolomics data, we were particularly interested in three types of comparisons in the context of Type 1 Diabetes (FIG. 1d): over-all differences in longitudinal profiles, age-based comparison between progressors and non-progressors, and metabolite profile changes related to associated with emergence of autoimmunity.

Lipidomic Analysis Reveals Age as the Main Confounding Factor

We performed lipidomics analysis on all selected 1039 samples using the UPLC-MS platform. While the data processing generated large number of unidentified peaks, the data analysis was limited to 186 lipid molecular species identified across all batches. In order to explore the data structure and identify the main confounding factors affecting the lipid profiles, we performed the Sammon's non-linear mapping⁹, which maps the samples nonlinearly from high (e.g. 186)-dimensional space to low-dimensional space, while aiming to preserve profile (e.g. Euclidean) distances between the samples. Compared to more commonly utilized linear methods such as Principle Components Analysis¹⁰, the Sammon's method is superior in ability to extract information from highly interdependent features¹¹ and is a more direct way to visualize similarities of profiles from the raw data.

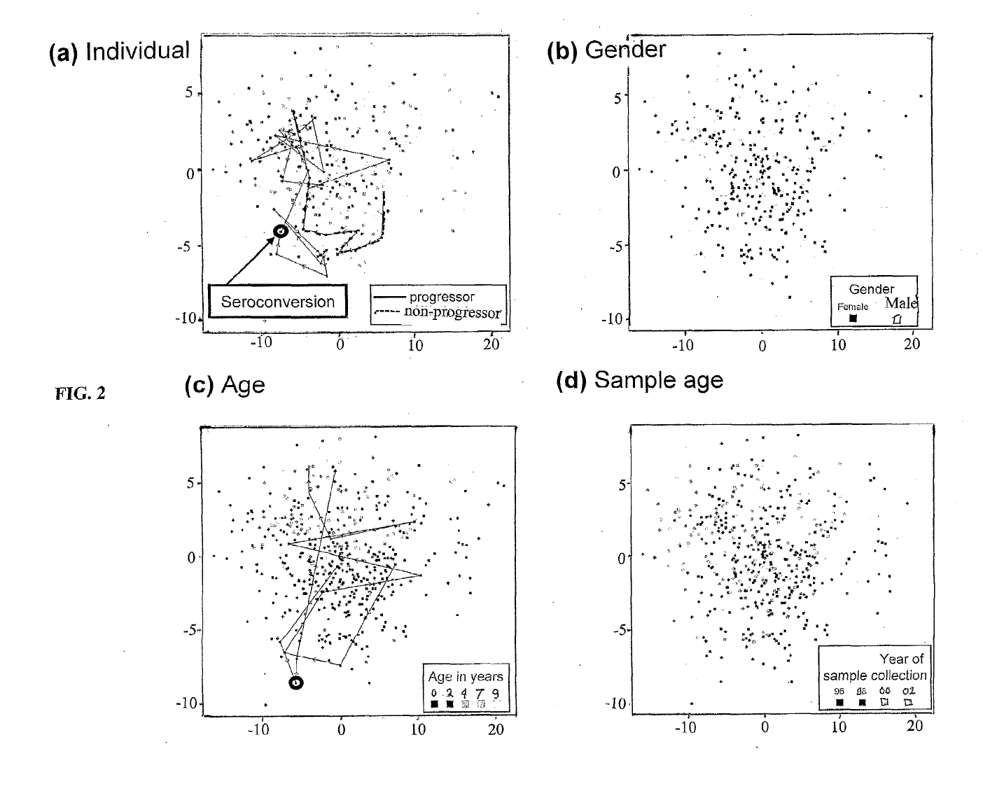

The FIG. 2 displays the results of Sammon's mapping of the Oulu DIPP batch for four potential confounding factors: individual ID, gender, age, and sample age. It is evident that neither sample age nor gender are major factors affecting similarities of lipid profiles. However, the profiles do cluster on age (FIG. 2c), i.e. the lipid profiles of children at early age are more similar to each other than to their profiles at later stage. This can be expected both due to diet, which varies with age and is generally more uniform at early age, as well as due to significant changes of childrens' metabolism due to their development. Interestingly, between-individual differences can also be detected (FIG. 2d).

Early Age Serum Lipidome Differences Between Progressors and Non-Progressors Prior to Autoimmunity

In order to examine the feasibility of early age disease prediction, we performed multivariate cross-sectional analysis for specific age groups using partial least squares discriminant analysis¹². The PLS/DA models were developed independently for the three batches analyzed.

We found that there are clear differences between progressors and non-progressors already at age of one year, and that these differences are attributed to the same or related molecular species across all three batches. We also applied the model developed for DIPP Turku batch to select most important lipid molecular species based on VIP analysis. A new PLS/DA model was developed based on selected lipid species and applied to the other two batches, and we found that it correctly predicts the onset of diabetes.

Our results imply the lipidomics strategy, possibly in combination with prior genetic screening, may markedly antedate and improve accuracy of defining which children will later progress to autoimmunity and over Type 1 Diabetes.

Consistent Differences in Plasmalogen Molecular Species Between Cases and Controls

The early age differences found in serum lipid profiles suggest disease-related events occur much earlier than previously thought. In order to examine within-individual lipid level changes in time and so determine consistency of observed changes in serum lipidome, we studied the longitudinal profiles of each identified lipid molecular species. Noticeably, we found decreased level of multiple choline plasmalogen molecular species in children who later progressed to autoimmunity and overt Type 1 Diabetes already at an early age, i.e. markedly before development of signs of autoimmunity (FIG. 3). The differences persist for all ages and the plasmalogen levels do not appear to be affected by the emergence of the disease itself (last time point for progressors).

Plasmalogens, a sub-class of ether linked phospholipids, have been previously implicated in protection against oxidative damage¹³⁻¹⁵. Reactive oxygen species (ROS) have been proposed to play an important role in β-cell destruction and it has been shown that exposure of pancreatic islets to cytokines increases ROS production and leads to oxidative damage to β cells¹⁶. The β cells are particularly susceptible to oxidative damage as they contain low levels of antioxidant enzymes¹⁷.

Antioxidant therapies have been proposed as a possible strategy to prevent diabetes¹⁸, but the results so far are confusing¹⁹. Our results suggest that the ability to protect against oxidative damage plays a major role in Type 1 Diabetes pathogenesis, not the ROS generation itself.

It is known that the last parts of plasmalogen synthesis are localized the endoplasmic reticulum (ER)²⁰. There is substantial evidence from in vivo studies that ER stress plays an important role in disease pathogenesis.

Cord Blood Analysis Reveals Decreased Phosphocholine Levels in Children who Later Progressed to Diabetes

The early age differences in lipid phenotype raise the possibility that the metabolic phenotypes of children who later progressed to diabetes differ already at birth. For that purpose we examined the cord blood samples of 39 children, of which 15 later progressed to Type 1 diabetes until age 12 or earlier. The children were born in Turku, but were not the same as studied in previous analyses.

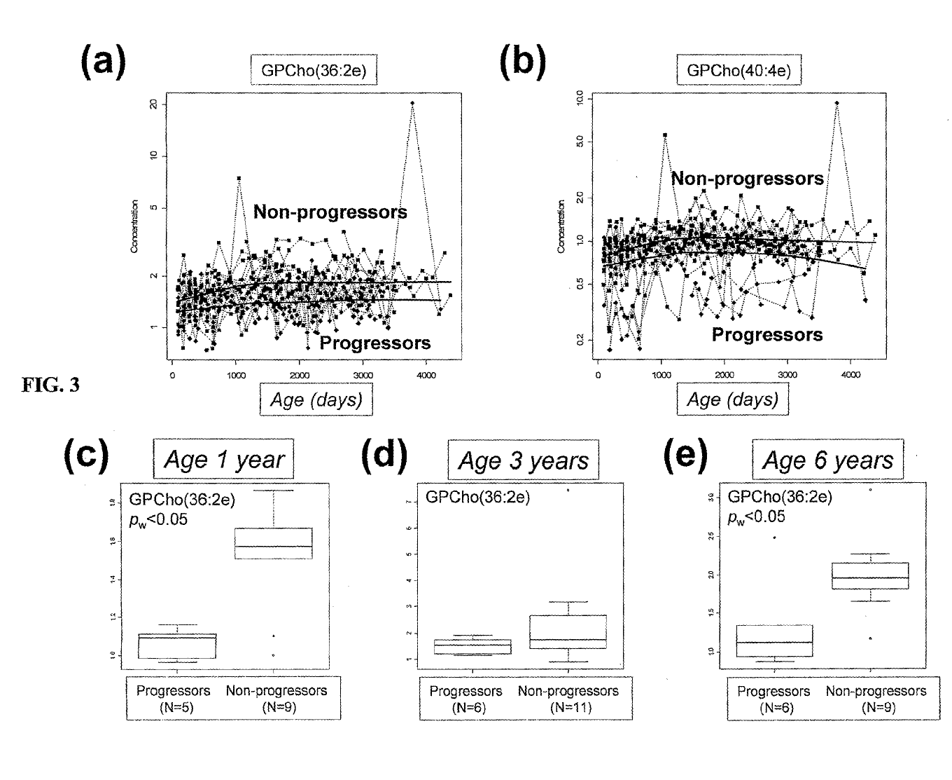

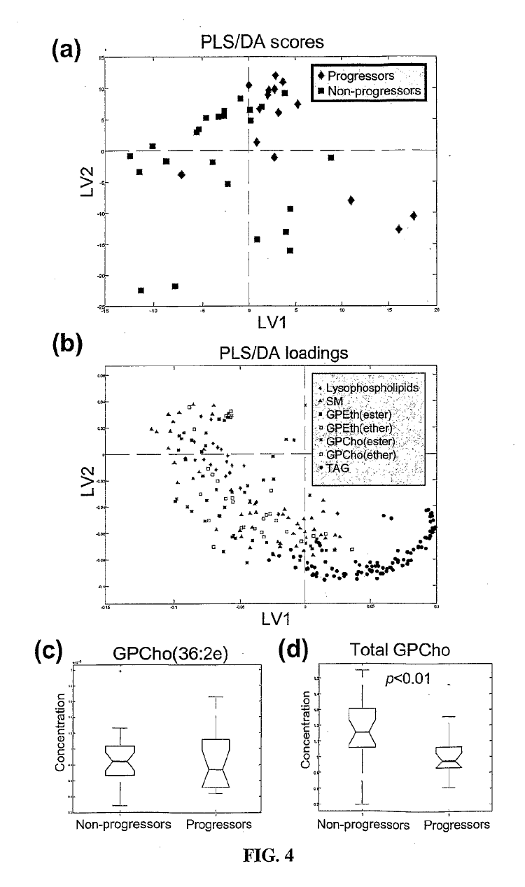

The multivariate analysis identified two major factors affecting the grouping of samples (FIG. 4). The increased levels of triacylglycerols affected both the progressors and non-progressors. However, another major factor that appears to discriminate the majority of the samples from the two groups is change in phospholipids levels (FIGS. 4a and 4b). The plasmalogen species GPCho(36:2e) found to be downregulated in progressors already at an early age does not differ significantly between the groups (FIG. 4c). However, the total ester linked phosphocholine levels, the most abundant phospholipids species in serum, are significantly downregulated in progressors already at birth (FIG. 4d).

Seroconversion

We also investigated whether the observed lipid profile changes associate with the emergence of autoimmunity. For that purpose we compared serum lipid profiles within the period of 6 months prior to seroconversion and the period immediately following the seroconversion.

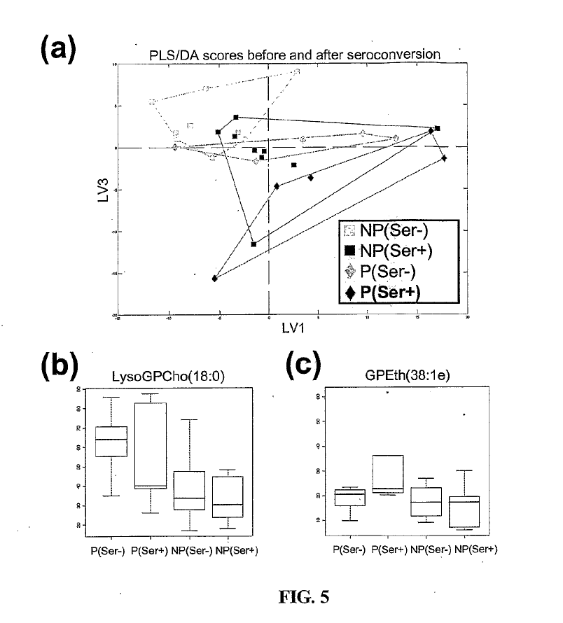

The choline plasmalogen levels of progressors, as already shown in FIG. 3, did not alter with emergence of autoimmunity. The major factor prior to seroconversion in progressors is upregulated lysophosphatidylcholine (FIG. 5). The lysophosphatidylcholine (LysoPC) has been associated with inflammation²¹, therefore suggesting an existence of an event leading to inflammation prior to autoimmunity. Importantly, LysoPC has been shown to enhance cytokine production²². The specific upregulation of LysoPC is transient; it appears only within the short time interval.

The changes following the seroconversion are dominated by increase in ethanolamine plasmalogen levels (FIG. 5). This suggests the increase in these ether linked phospholipids is a normal systemic response to increase in oxidative damage.

In summary, the longitudinal serum lipid profiles of children who later developed Type 1 diabetes revealed several successive events leading to autoimmunity and the disease (FIG. 6), suggesting a key role of phospholipids metabolism in early disease pathogenesis. The emerging picture of disease pathogenesis reveals complex interplay of pro-pathogenic factors and the compensatory responses.

Feasibility of Prediction of Type 1 Diabetes at an Early Age

Lipidome changes observed suggest that disease prediction using metabolic profiling prior to seroconversion may be feasible. A classification algorithm was therefore developed based on the extended lipid profiles from the randomly selected subset of 60% of progressors and non-progressors. Based on known longitudinal profile variation and no observed dependence on confounding factors, only ether phospholipids were considered as potential biomarkers. Best disease prediction was observed at an early age, with the optimal biomarker at age 1.5 year (range 0.5-2.5 years) consisting of GPCho(O-18:1/16:0) molecular species (Table 1). The classification rule for progressors consisted of a requirement that the lipid concentration lies below 4.09 μmol/L.

The performance of the classifier was assessed by testing the null hypothesis that the test outcome shows no association with onset of type 1 diabetes. In order to control for bias, test and training sets were randomly selected 1000 times. For each selection, lipid-specific classification thresholds were determined on the training set and the classification accuracy was assessed in the test set. Binomial distributions were used to exactly calculate the P-values corresponding to probabilities of obtaining at least the observed number of true positives (TP) or at most the observed number of false positives (FP) if the random classifier (TP=FP) corresponding to the null hypothesis was used. Summary statistics, median and 80% confidence interval of each variable are reported.

TABLE 1
Performance of the classifier consisting of a single ether
phosphatidylcholine GPCho(O-18:1/16:0). A subject is classified as a progressor if
the ether phosphatidylcholine concentration is below 4.1 μmol/L, with 90% CI =
[4.0 μmol/L, 4.7 μmol/L]. Autoantibody positive samples are excluded from the
analysis.

| | | | | | | Odds |
|---|---|---|---|---|---|---|
| Sample series | Age range | TP/P | P (TP) | FP/N | P (FP) | ratio |
| Training set | Average overall samples | 12 [9, 14]/22 | 1.5 × 10⁻⁴ | 7 [5, 11]/40 | 4.2 × 10⁻⁶ | 12.2 [4.1, 46.9] |
| | per subject within 0.5-2.5 | | | | | |
| | year age period | | | | | |
| Test set | Average overall samples | 7 [3, 9]/14 | 0.051 | 6 [2, 12]/26 | 0.014 | 3.0 [1.1, 8.3] |
| | per subject within 0.5-2.5 | | | | | |
| | year age period | | | | | |
| Test set | One sample per subject, | 7 [4, 10]/14 | 0.036 | 6 [3, 11]/26 | 0.006 | 3.2 [1.3, 8.3] |
| | closest to age 1.5 year | | | | | |

TP, number of true positives;
P, number of positives (i.e., progressors);
P (TP), probability number of true positives is greater than TP by chance;
FP, number of false positives;
N, number of negatives (i.e., non-progressors);
P (FP), probability number of false positives is less than FP by chance.
90% confidence intervals for TP, FP, and Odds ratios, based on 1000 random selections of test and training sets, are shown in brackets.

Methods

Serum collection. Vena blood samples were collected from children during the years 1994-2004. The samples were taken various times through the day without fasting. Blood samples were taken by venous withdrawal using a needle and BD Vacutainer® Plastic Tubes or Vacutainer® Plus Plastic Tubes. (BD Vacutainer® SSTTM Tubes contain spray-coated silica and a polymer gel for serum separation.) The tubes were left at RT 30-60 min to coagulate. Serum was separated by centrifugation at 1300 rcf for 10 min at room temperature. The serum samples were stored in small plastic tubes at −80° C.

Lipidomics. An aliquot (10 μl) of an internal standard mixture containing 11 lipid classes, and 0.05M sodium chloride (10 μl) was added to serum samples (10 μl) and the lipids were extracted with chloroform/methanol (2:1, 100 μl). After vortexing (2 min), standing (1 hour) and centrifugation (10000 RPM, 3 min) the lower layer was separated and a standard mixture containing 3 labelled standard lipids was added (10 μl) to the extracts. The internal standard mixture contained the following lipid compounds (pg/ml) with heptadecanoic acid (C17:0) as the esterified fatty acid:

D-erythro-Sphingosine-1-Phosphate (9.3 μg/ml; C17 Base, Avanti Polar Lipids),

1-Heptadecanoyl-2-Hydroxy-sn-Glycero-3-Phosphocholine (8.8 μg/ml; Avanti Polar Lipids),

1-Monoheptadecanoin (rac) (9.3 μg/ml; Larodan Fine Chemicals),

1,2-Diheptadecanoyl-sn-Glycero-3-[Phospho-rac-(1-glycerol)] (9.6 μg/ml; Avanti Polar Lipids),

N-Heptadecanoyl-D-erythro-Sphingosine (9.2 μg/ml; Avanti Polar Lipids),

1,2-Diheptadecanoyl-sn-Glycero-3-[Phospho-L-Serine] (8.6 μg/ml; Avanti Polar Lipids),

1,2-Diheptadecanoyl-sn-Glycero-3-Phosphocholine (9.9 μg/ml; Avanti Polar Lipids),

1,2-Diheptadecanoyl-sn-Glycero-3-Phosphate (8.5 μg/ml; Avanti Polar Lipids),

1,2-Diheptadecanoyl-sn-Glycero-3-Phosphoethanolamine (8.9 μg/ml; Avanti Polar Lipids),

1,2-Diheptadecanoin (rac) (10.2 μg/ml; Larodan Fine Chemicals) and

Triheptadecanoin (10.4 μg/ml; Larodan Fine Chemicals).

The labeled standard mixture consisted of

L-α-Lysophosphatidylcholine-Palmitoyl-D3 (9.3 μg/ml; Larodan Fine Chemicals),

1,2-Dipalmitoyl-D6-sn-Glycerophosphatidylcholine (11.7 μg/ml; Larodan Fine Chemicals) and

Tripalmitin-1,1,1-¹³C3 (10.0 μg/ml; Larodan Fine Chemicals).

When analysing the first 232 samples (Batch 1), only one standard mixture (25 μl) containing triheptadecanoin (0.804 mg/ml; Larodan Fine Chemicals) and 1,2-dipentadecanoyl-sn-glycero-3-phosphocholine (0.304 mg/ml; Larodan Fine Chemicals) was added to serum samples (15 μl) before lipid extraction with chloroform/methanol (2:1, 100 μl).

Lipid extracts were analysed on a Waters Q-Tof Premier mass spectrometer combined with an Acquity Ultra Performance LC™ (UPLC). The column, which was kept at 50° C., was an Acquity UPLC™ BEH C18 10×50 mm with 1.7 μm particles. The binary solvent system included A. water (1% 1M NH₄Ac, 0.1% HCOOH) and B. LC/MS grade (Rathburn) acetonitrile/isopropanol (5:2, 1% 1M NH₄Ac, 0.1% HCOOH). The gradient started from 65% A/35% B, reached 100% B in 6 min and remained there for the next 7 min. The total run time including a 5 min re-equilibration step was 18 min. The flow rate was 0.200 ml/min and the injected amount 0.75 μl. The temperature of the sample organizer was set at 10° C. The lipid profiling was carried out on Waters Q-Tof Premier mass spectrometer using ESI+ mode. The data was collected at mass range of m/z 300-1200 with a scan duration of 0.2 sec. For the last samples the scan time was changed to 0.02 sec. The source temperature was set at 120° C. and nitrogen was used as desolvation gas (800 L/h) at 250° C. The voltages of the sampling cone and capillary were 39 V and 3.2 kV, respectively. Reserpine (50 μg/L) was used as the lock spray reference compound (5 μl/min; 10 sec scan frequency). Tandem mass spectrometry was used for the identification of selected molecular species of lipids. MS/MS runs were performed by using ESI+ mode, collision energy ramp from 15 to 30 V and mass range starting from m/z 150. The other conditions were as shown above.

Processing and analysis of metabolomics data. Data was processed using MZmine software version 0.60^{23,24}. Metabolites were identified using internal spectral library.

Partial least squares discriminant analysis (PLS/DA)^{12,25} was utilized as a supervised modeling method using SIMPLS algorithm to calculate the model²⁶. Venetian blinds cross-validation method²⁷ and Q² scores were used to develop the models. Top loadings for latent variables associated with drug specific effects were reported. The VIP (variable importance in the projection) values²⁸ were calculated to identify the most important molecular species for the clustering of specific groups. Multivariate analyses were performed using Matlab version 7.2 (Mathworks, Inc.) and the PLS Toolbox version 4.0 Matlab package (Eigenvector Research, Inc.).

Other Serum Metabolites (i.e. Non-Phospholipids) Found in Cord Blood

Methods:

The serum samples were prepared as follows: 400 μl methanol and 10 μl 250 ppm d3-palmitic acid (internal standard) were added to a 25 μl serum sample. The samples were vortexed for 30 seconds. After 30 minutes the samples were centrifuged for 3 min at 10000 rpm. Supernatant was moved to a GC vial and evaporated to dryness under nitrogen. The samples were silylated with 20 μl MOX (45° C., 60 min) and 20 μl MSTFA (45° C., 60 min). 5 μl of retention index solution was added to samples (600 ppm C11, C15, C17, C21 and C25 alkanes).

Instrument:

The instrument used was a Leco Pegasus 4D GCxGC-TOF mass spectrometer with Agilent 6890N GC and Combi PAL autosampler. The instrument parameters were as follows:

2 μl split injection 1:20 for serum samples.

First column: RTX-5, 10 m×180 μm×0.20 μm

Second column: BPX-50, 1.10 m×100 μm×0.10 μm

Helium 35.33 psig constant pressure

Temperature Programmes:

Primary oven: Initial 50° C., 1 min.→280° C., 7° C./min, 5 min.

Secondary oven: +10° C. above primary oven temperature.

Second dimension separation time 4 s.

MS measurement 40-700 amu, 100 spectra/s.

Method Characteristics:

The performance characteristics of GCxGC-TOF have been tested with three pure, non-extracted, reference compounds. All compounds were made in eight concentration levels between 10 and 30000 ng/sample.

L-Threonine:

Linear range: 7.4-2200 ng

Correlation coefficient (at linear range): 0.99975

Relative standard deviation (8 samples, 7440 ng): 7.60%

S/N at lowest concentration 7.4 ng: 56.6

Lauric Acid:

Linear range: 10-30000 ng

Correlation coefficient: 0.99737

Relative standard deviation (7 samples, 10100 ng): 2.61%

S/N at lowest concentration 10.1 ng: 115.3

Cholesterol:

Linear range: 10-30000 ng

Correlation coefficient: 0.99999

Relative standard deviation (7 samples, 10000 ng): 2.89%

S/N at lowest concentration 10.0 ng: 62.7

Data Processing:

ChromaTof software was used for within-sample data processing, and in house made software was used for alignment and peak matching across samples. The peaks were filter based on number of detected peaks in the total profile of 36 samples (set to 12 peaks found minimum) and based on identity match in the database (similarity index threshold=800).

Results: The results are shown in Table 2 below. The column Fold (median) shows the ratio of median value of metabolite levels of children who progressed to type 1 diabetes and median value for children who remained autoantibody negative during the follow-up (non-progressors). p(Wilcoxon) is the p value based on Wilcoxon rank sum test comparing the two groups. The column Fold (mean) shows the ratio of mean value of metabolite levels of children who progressed to type 1 diabetes and mean value for children who remained autoantibody negative during the follow-up (non-progressors). p(ttest) is the p value based on two-sided t-test comparing the two groups.

Table 2 (will be added in landscape format)

TABLE 2

| Name | Fold (median) | p(Wilcoxon) | Fold (mean) | p(ttest) |
|---|---|---|---|---|
| Tryptophan, bis(trimethylsilyl)- | 0.68 | 0.0224 | 0.52 | 0.0197 |
| Ribitol, 1,2,3,4,5-pentakis-O-(trimethylsilyl)- | 0.45 | 0.0369 | 0.42 | 0.0256 |
| Pentanedioic acid, 2-[(trimethylsilyl)oxy]-, bis(trimethylsilyl) ester | 0.36 | 0.0205 | 0.57 | 0.0258 |
| Glycine, N,N-bis(trimethylsilyl)-, trimethylsilyl ester | 0.86 | 0.0694 | 0.72 | 0.0272 |
| Eicosanoic acid, trimethylsilyl ester | Inf | 0.0267 | [symbol] | 0.0296 |
| 1,2,3-Propanetricarboxylic acid, 2-[(trimethylsilyl)oxy]-, tris(trimethylsilyl) | 0.60 | 0.0184 | 0.70 | 0.0298 |
| MYRISTOLEIC ACID 1TMS | 0.00 | 0.0587 | 0.30 | 0.0330 |
| MANNITOL TMS | 0.45 | 0.0248 | 0.39 | 0.0332 |
| Creatinine enol N1,N3,O-tris(trimethylsilyl) | 0.82 | 0.0923 | 0.77 | 0.0476 |
| Butanedioic acid, bis(trimethylsilyl) ester | 0.24 | 0.0419 | 0.46 | 0.0494 |
| HEPTANOIC ACID TMS | 1.19 | 0.0390 | [symbol] | 0.1719 |
| 2-KETOGLUTARIC ACID-METHOXIME-MONOTMS | 0.48 | 0.0314 | 0.66 | 0.2604 |

[symbol]

indicates data missing or illegible when filed

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### REFERENCES

1. Kupila, A. et al. Feasibility of genetic and immunological prediction of Type I diabetes in a population-based birth cohort. Diabetologia 44, 290-297 (2001).
2. Oresic, M., Vidal-Puig, A. & Hanninen, V. Metabolomic approaches to phenotype characterization and applications to complex diseases. Expert Rev. Mol. Diagn. 6, 575-585 (2006).
3. Clayton, A. T. et al. Pharmaco-metabonomic phenotyping and personalized drug treatment. Nature 440, 1073-1077 (2006).
4. Robosky, L. C. et al. Metabonomic identification of two distinct phenotypes in Sprague-Dawley (Crl:CD(SD)) rats. Toxicol. Sci. 87, 277-284 (2005).
5. Knip, M. et al. Environmental triggers and determinants of Type 1 Diabetes. Diabetes 54, S125-136 (2005).
6. Raamsdonk, L. M. et al. A functional genomics strategy that uses metabolome data to reveal the phenotype of silent mutations. Nat. Biotechnol. 19, 45-50 (2001).
7. Oresic, M. et al. Phenotype characterization using integrated gene transcript, protein and metabolite profiling. Appl. Bioinformatics 3, 205-217 (2004).
8. Simell, O. et al. Special Turku Coronary Risk Factor Intervention Project for Babies (STRIP). Am J Clin Nutr 72, 1316S-1331(2000).
9. Sammon Jr., J. W. A nonlinear mapping for data structure analysis. IEEE Trans. Comp. C-18, 401-409 (1969).
10. Jackson, J. E. User's guide to principal components (John Wiley & Sons, New York, N.Y., 1991).
11. De Backer, S., Naud, A. & Scheunders, P. Non-linear dimensionality reduction techniques for unsupervised feature extraction. Pattern Recog. Lett. 19, 711-720 (1998).
12. Barker, M. & Rayens, W. Partial least squares for discrimination. J. Chemometrics 17, 166-173 (2003).
13. Engelmann, B. Plasmalogens: targets for oxidants and major lipophilic antioxidants. Biochem. Soc. Trans. 32, 147-150 (2004).
14. Zoeller, R. A. et al. Plasmalogens as endogenous antioxidants: somatic cell mutants reveal the importance of the vinyl ether. Biochem J. 338, 769-776 (1999).
15. Zoeller, R. A. et al. Increasing plasmalogen levels protects human endothelial cells during hypoxia. Am J Physiol Heart Circ Physiol 283, H671-679 (2002).
16. Tabatabaie, T., Vasquez-Weldon, A., Moore, D. R. & Kotake, Y. Free radicals and the pathogenesis of Type 1 Diabetes: {beta}-cell cytokine-mediated free radical generation via cyclooxygenase-2. Diabetes 52, 1994-1999 (2003).
17. Lenzen, S., Drinkgem, J. & Tiedge, M. Low antioxidant enzyme gene expression in pancreatic islets compared with various other mouse tissues. Free Radic. Biol. Med. 20, 463-466 (1996).
18. Piganelli, J. D. et al. A metalloporphyrin-based superoxide dismutase mimic inhibits adoptive transfer of autoimmune diabetes by a diabetogenic T-cell clone. Diabetes 51, 347-355 (2002).
19. Li, X., Chen, H. & Epstein, P. N. Metallothionein and catalase sensitize to diabetes in Nonobese Diabetic Mice: Reactive oxygen species may have a protective role in pancreatic {beta}-cells. Diabetes 55, 1592-1604 (2006).
20. Nagan, N. & Zoeller, R. A. Plasmalogens: biosynthesis and functions. Prog. Lipid Res. 40, 199-229 (2001).
21. Murphy, A. A., Santanam, N., Morales, A. J. & Parthasarathy, S. Lysophosphatidyl choline, a chemotactic factor for monocytes/T-lymphocytes is elevated in endometriosis. J. Clin. Endocrin. Metab. 83, 2110-3 (1998).
22. Takabe, W. et al. Lysophosphatidylcholine enhances cytokine production of endothelial cells via induction of L-type amino acid transporter 1 and cell surface antigen 4F2. Arterioscler Thromb Vasc Biol 24, 1640-1645 (2004).
23. Katajamaa, M. & Oresic, M. Processing methods for differential analysis of LC/MS profile data. BMC Bioinformatics 6, 179 (2005).
24. Katajamaa, M., Miettinen, J. & Oresic, M. MZmine: toolbox for processing and visualization of mass spectrometry based molecular profile data. Bioinformatics 22, 634-636 (2006).
25. Geladi, P. & Kowalski, B. R. Partial least-squares regression: a tutorial. Anal. Chim. Acta 185, 1-17 (1986).
26. de Jong, S. SIMPLS: An alternative approach to partial least squares regression. Chemometr. Intell. Lab. Syst. 18, 251-263 (1993).
27. Wise, B. M. et al. PLS Toolbox 3.5 for use with Matlab (Eigenvector Research Inc., Manson, Wash., 2005).
28. Wold, S., Esbensen, K. & Geladi, P. Principal Component analysis.

Chemometr. Intell. Lab. Syst. 2, 37-52 (1987).

1-21. (canceled)

22. A method for diagnosing a child's susceptibility for developing type I diabetes, wherein said method comprises
i) determining the concentration of at least one serum metabolite in the child to be diagnosed,
ii) comparing the serum concentration of said metabolite to the serum concentration of the same metabolite in a control group of healthy children,
iii) using a concentration difference between the child to be diagnosed and the control group as a biomarker indicative of the child's susceptibility for developing type I diabetes.

23. The method according to claim 22, wherein the age of the child to be diagnosed is the same or approximately the same as that of the control group.

24. The method according to claim 22, wherein the biomarker is a metabolite protecting against oxidative stress and/or inflammation, and a decreased concentration thereof in the child to be diagnosed, compared to the control group of healthy children, is indicative of the child's susceptibility for developing type I diabetes.

25. The method according to claim 24 wherein the biomarker is a phospholipid, an acid or a derivative thereof, a ketone, or an alcohol.

26. The method according to claim 25, wherein the biomarker is total phospholipids.

27. The method according to claim 25, wherein the biomarker is one or more ester linked phosphocholines.

28. The method according to claim 25, wherein the biomarker is total ester linked phosphocholines.

29. The method according to claim 22, wherein the biomarker is determined in newborn children.

30. The method according to claim 28, wherein the child is a newborn child and child's level of total ester linked phosphocholines being about 80% or less of the mean level for the control group is used as indicative of the child's susceptibility for developing type I diabetes.

31. The method according to claim 25, wherein the biomarker is one or more ether linked phosphocholine, or an ethanolamine plasmalogen.

32. The method according to claim 31, wherein the ether linked phosphatidylcholine is selected from the group consisting of GPCho (36:2e), GPCho (38:1e), GPCho (38:5e), GPCho (40:4e), CPCho (O-18:1/16:0), CPCho (O-18:1/16:1), CPCho (O-16:0/20:4), CPCho (O-18:1/20:4) and CPCho (O-18:0/18:2), and the determination thereof is made at a child age ranging from newborn to six years' age.

33. The method according to claim 31, wherein the ethanolamine plasmalogen is GPEtn (O-18:1(1Z)/20:4), and the determination thereof is made at a child age ranging from newborn to six years' age.

34. The method according to claim 25, wherein the biomarker is selected from a group consisting of tryptophan, ribitol, pentanedioic acid, glycine, eicosanoic acid, 1,2,3-propanetricarboxylic acid, myristoleic acid, mannitol, creatinine, butanedioic acid, heptanoic acid and 2-ketoglutaric acid methoxime.

35. The method according to claim 22, wherein the determination of the serum metabolite is made at several ages of the child and the result is compared to control groups of the same age as the child to be diagnosed.

36. The method according to claim 35, wherein several serum metabolites are determined for the child to be diagnosed, and the levels are compared to the levels of said metabolites for control groups.

37. The method according to claim 22, wherein the genetic risk for development of type 1 diabetes and/or the emergence of autoimmunity also is determined.

38. The method according to claim 37, wherein the genetic risk for development of type 1 diabetes and/or the emergence of autoimmunity are followed by metabolite markers as progressive disease susceptibility detection.

39. The method according to claim 37, wherein the emergence of autoantibody markers in combination with the decreased ether linked phosphocholine levels are determined to identify individuals at higher risk of developing type 1 diabetes.

40. A method for prevention of the onset of type 1 diabetes in a child, said child having been diagnosed according to claim 22, as susceptible for developing type I diabetes, said method comprising subjecting said child to one or more measures preventing the onset of diabetes.

41. The method according to claim 40, wherein the preventing measure is a nutritional intervention, an antioxidant therapy, or a stimulation of the biochemical synthesis of choline plasmalogens in the child, or any combination of said measures.

42. The method according to claim 41, wherein the nutritional intervention is a choline supplement in the mother's diet, a choline supplement in the child's diet or a choline plasmalogen supplement in the child's diet.
